(19) United States
(12) Patent Application Publication
(10) Pub. No.: US 2017/0105699 A1
(43) Pub. Date: Apr. 20, 2017

(54) ULTRASONIC DIAGNOSTIC DEVICE

(71) Applicant: Hitachi, Ltd., Tokyo (JP)

(72) Inventors: Kagami MIYAJI, Kanagawa (JP); Keiichi ITATANI, Kanagawa (JP); Hajime SAKASHITA, Tokyo (JP); Tomohide NISHIYAMA, Tokyo (JP); Yoshinori SEKI, Tokyo (JP)

(73) Assignee: Hitachi, Ltd., Tokyo (JP)

(21) Appl. No.: 15/300,601

(22) PCT Filed: Mar. 11, 2015
(86) PCT No.: PCT/JP2015/057151

§ 371 Date: Sep. 29, 2016

(30) Foreign Application Priority Data
Mar. 31, 2014 (JP) 2014-070936

(51) Int. Cl. A61B 8/06; A61B 8/08; G01S 15/89; A61B 8/00

(52) U.S. Cl. CPC A61B 8/06; A61B 8/4488; A61B 8/488; A61B 8/5223; A61B 8/461; A61B 8/54; G01S 15/8984

(57) ABSTRACT

A transmission reception unit forms a transmission beam by outputting a transmission signal to each of a plurality of vibrating elements provided in a probe and further forms a reception beam on the basis of a plurality of wave reception signals obtained from the plurality of vibrating elements. Thus, an ultrasonic beam (transmission beam and reception beam) is scanned in a scan surface. A velocity vector computation unit forms a two-dimensional velocity vector distribution in the scan surface from information regarding velocity of blood flow in an ultrasonic beam direction. A vortex detection unit tracks a flow of a fluid on the basis of the two-dimensional velocity vector distribution obtained by the velocity vector computation unit and detects a vortex in the fluid on the basis of whether or not the flow of the fluid satisfies a recurrence condition.

### TECHNICAL FIELD

The present invention relates to an ultrasonic diagnostic device, and more particularly to a technique of obtaining diagnosis information regarding fluid.

### BACKGROUND

Techniques known to the art include obtaining diagnosis information regarding fluid based on a received signal obtained by transmitting and receiving ultrasonic waves with respect to the fluid such as a blood flow. Patent Document 1, for example, describes a technique of obtaining, based on a received signal (echo data) obtained by transmitting and receiving ultrasonic waves with respect to fluid within an organism, a two-dimensional velocity vector regarding fluid at a plurality of points within an observation plane. It is possible to extract, from a distribution of the two-dimensional velocity vectors at a plurality of points within an observation plane, diagnosis information such as a streamline representing a flow of fluid. Application of such information to diagnosis of a heart, for example, is expected.

In diagnosis of a heart, attention may be focused on a vortex of a blood flow within the heart. For example, a user, such as a doctor, may see a distribution of two-dimensional velocity vectors or a streamline regarding a blood flow displayed on an ultrasonic diagnostic device to visually confirm the state of a vortex, for example.

### CITATION LIST

### Patent Literature

Patent Document 1: JP-2013-192643 A

### SUMMARY

### Technical Problem

Research and development has been repeated by the inventors of the present application, in consideration of the background art described above, concerning the techniques of obtaining diagnosis information regarding fluid such as a blood flow by utilizing ultrasonic waves.

The present invention was achieved in the process of the research and development, and is directed to providing a technique of detecting a vortex in fluid by utilizing ultrasonic waves.

### Solution to Problem

In accordance with one aspect of the invention, an ultrasonic diagnostic device includes a probe configured to transmit and receive an ultrasonic wave; a transmitter and receiver unit configured to control the probe to obtain a received signal of an ultrasonic wave from within an organism; a vector computation unit configured to obtain, based on the received signal of an ultrasonic wave, a distribution of a motion vector concerning fluid within the organism; and a vortex detection unit configured to track a flow of the fluid based on the distribution of a motion vector and, based on the flow of the fluid satisfying a recurrence condition, detect a vortex within the fluid.

In the above configuration, a motion vector refers to vector information regarding a motion of fluid, and specifically includes, for example, a velocity vector indicating the velocity and direction of each portion within fluid and a shifting vector indicating an amount and direction of shift of each portion. The distribution of motion vectors can be obtained by utilizing the technique described, for example, in Patent Document 1 (two-dimensional velocity vector distribution), but may also be obtained by utilizing other known techniques.

The recurrence condition refers to a condition for evaluating the state of a flow of fluid, and may be, for example, a condition used for selecting, as a vortex, a flow of fluid moving toward a distant location and then returning back to the original location or near the original location. For example, when the result of tracking of a flow of fluid satisfies the recurrence condition, the flow is determined as a vortex.

The above configuration of the device for detecting a vortex within fluid based on whether or not the flow of the fluid satisfies the recurrence condition can free users from complicated operations for detecting a vortex, for example, and, in some embodiments, can eliminate user operations for detecting a vortex.

In some preferable specific examples, the vortex detection unit may be configured to track, for each of a plurality of start points, a flow of fluid in accordance with the distribution of a motion vector from each start point and determine, based on the flow of fluid being tracked from each start point satisfying the recurrence condition, that the flow is a vortex.

In some preferable specific examples, the vortex detection unit may be configured to track the flow of fluid from each start point to obtain a streamline and determine whether or not the flow is a vortex by using the streamline, in accordance with a recurrence condition based on a distance between the start point and a point on the streamline.

In some preferable specific examples, the vortex detection unit may be configured to further determine, when the flow of fluid being tracked from each start point is a vortex, whether or not a flow of fluid being tracked from each of a plurality of start points outside the vortex is a vortex, and determine an outer edge of the vortex based on a flow of fluid obtained from an outermost start point which is determined to correspond to a vortex.

In some preferable specific examples, the vortex detection unit may be configured to determine a noted point within a vortex as a center point of the vortex, based on opposing motion vectors, among a plurality of motion vectors enclosing the noted point, being directed to opposite directions.

In some preferable specific examples, the vortex detection unit may be configured to determine a noted point detected within a two-dimensional plane as a center point of a vortex, based on motion vectors adjacent to each other in a vertical direction with respect to the noted point being directed in opposite directions and motion vectors adjacent to each other in a horizontal direction with respect to the noted point being directed in opposite directions.

In some preferable specific examples, the vortex detection unit may be configured to determine, upon detecting a plurality of vortexes having a center point at an identical location, a largest vortex among the plurality of vortexes as a vortex corresponding to the center point.

In accordance with another aspect, a fluid information processor includes a vector computation unit configured to obtain a distribution of a motion vector concerning fluid within an organism based on a received signal of an ultrasonic wave; and a vortex detection unit configured to track a flow of the fluid based on the distribution of a motion vector and detect a vortex within the fluid based on the flow of the fluid satisfying a recurrence condition.

The fluid information processor described above can be implemented by a computer. For example, it is possible to cause a computer to function as the fluid information processor described above using a program which causes the computer to implement a vector computation function to obtain a motion vector distribution regarding fluid within an organism based on a received signal of ultrasonic waves, and a vortex detection function to track the flow of fluid based on the motion vector distribution and detect a vortex within the fluid based on the flow of fluid satisfying the recurrence condition. The program may be stored in a computer-readable storage medium such as a disk and memory and provided to the computer via the storage medium or may be provided to the computer via an electric communication line such as the Internet.

### Advantageous Effects of Invention

The present invention provides a technique of detecting a vortex within fluid by utilizing ultrasonic waves. Because, in accordance with a preferable aspect of the present invention, for example, a vortex within a fluid is detected based on whether or not the flow of fluid satisfies the recurrence condition, the user can be freed from complicated operations for detecting a vortex. The need for user operations for detecting a vortex may be preferably eliminated.

### BRIEF DESCRIPTION OF DRAWINGS

FIG. 1 A diagram illustrating a whole structure of an ultrasonic diagnostic device according to an embodiment of the present invention.

FIG. 2 A diagram for explaining specific example processing for tracking flow of blood flow.

FIG. 3 A diagram illustrating a specific example arrangement of a plurality of start points SP.

FIG. 4 A diagram for explaining a specific example related to determination of a vortex.

FIG. 5 A diagram for explaining specific example processing for determining an outer edge of a vortex.

FIG. 6 A diagram illustrating a specific example center point of a vortex.

FIG. 7 A diagram illustrating a specific example related to display of a vortex.

### DESCRIPTION OF EMBODIMENTS

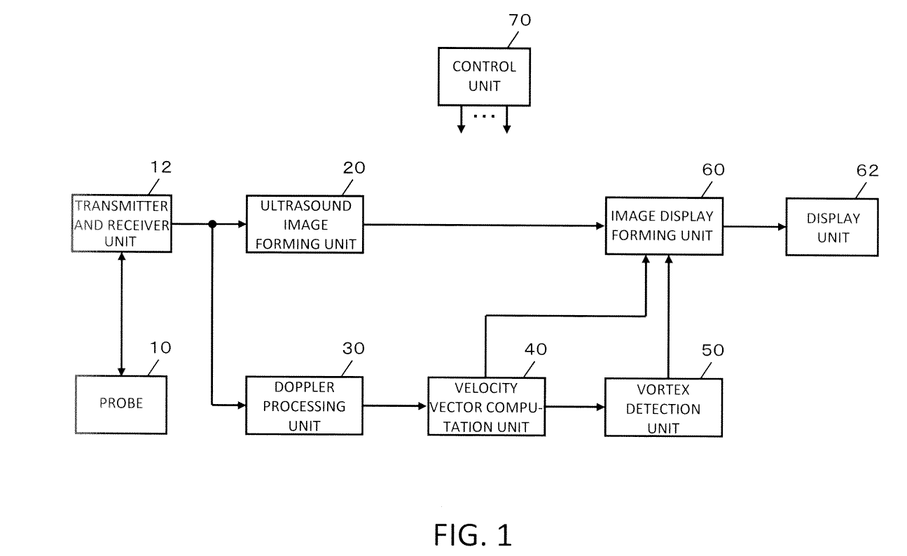

FIG. 1 is a diagram illustrating a whole structure of an ultrasonic diagnostic device according to a preferable embodiment of the present invention. The ultrasonic diagnostic device illustrated in FIG. 1 has a function to detect a vortex of fluid within an organism, and may particularly detect a vortex of a blood flow within a heart. Accordingly, detection of a vortex regarding a blood flow within a heart, which is example fluid to be diagnosed, will be described below.

A probe 10 is an ultrasound probe configured to transmit and receive ultrasonic waves to and from a space including a heart. The probe 10 includes a plurality of transducer elements, which are electrically scan-controlled to scan an ultrasound beam within a space including the heart. The probe 10 is held by a user (examiner) such as a doctor and is used in contact with a body surface of an examinee. Alternatively, the probe 10 may be inserted into a body cavity of the examinee for use.

A transmitter and receiver unit 12 has a function as a transmitting beam former and a received beam former. Specifically, the transmitter and receiver unit 12 outputs a transmitting signal to each of the plurality of transducer elements of the probe 10 to thereby form a transmitting beam, and further applies phase alignment and summation processing, for example, to a plurality of received signals obtained from the plurality of transducer elements to thereby form a received beam. Thus, an ultrasound beam (the transmitting beam and the received beam) is scanned within a scanning plane and a received signal is formed along the ultrasound beam.

An ultrasound image forming unit 20, based on the received signal of ultrasonic waves obtained from the scanning plane, forms image data of an ultrasound image. The ultrasound image forming unit 20 forms image data of a B-mode image concerning a cross section including a blood flow of the heart, for example.

A Doppler processing unit 30 measures a Doppler shift amount contained in the received signal obtained along the ultrasound beam. The Doppler processing unit 30 specifically measures a Doppler shift occurring within the received signal of the ultrasonic waves due to the blood flow by using known Doppler processing to obtain velocity information concerning the blood flow in the ultrasound beam direction.

A velocity vector computation unit 40, based on the velocity information concerning the blood flow in the ultrasound beam direction, forms a distribution of two-dimensional velocity vectors within the scanning plane. The distribution of two-dimensional velocity vectors within a scanning plane can be formed from one-dimensional velocity information along the ultrasound beam using various known methods.

As described in Patent Document 1 (JP 2013-192643 A), for example, the two-dimensional velocity vector of a blood flow at each location within the scanning plane may be obtained by using motion information of the cardiac wall, in addition to the velocity information concerning the blood flow in the ultrasound beam direction. Alternatively, the two-dimensional velocity vector may be formed by forming two ultrasound beams in different directions and obtaining velocity information from each of the two ultrasound beams.

The velocity vector computation unit 40 obtains a velocity vector for each of a plurality of sample points in a calculation coordinates system corresponding to a space in which the ultrasonic waves are transmitted and received. For example, the calculation coordinates system is represented by an xyz orthogonal coordinates system, and a velocity vector is obtained for each sample point within an xy plane corresponding to the scanning plane of ultrasonic waves to form the distribution of two-dimensional velocity vectors.

A vortex detection unit 50, based on the distribution of two-dimensional velocity vectors obtained by the velocity vector computation unit 40, tracks the flow of fluid, and detects a vortex within the fluid based on whether or not the flow of fluid satisfies a recurrence condition. Specific processing in the vortex detection unit 50 will be detailed below.

A display image forming unit 60 forms a display image based on the image data of an ultrasound image obtained by the ultrasound image forming unit 20, the two-dimensional velocity vectors obtained by the velocity vector computation unit 40, and the detection result of vortex in the vortex detection unit 50, for example. The display image forming unit 60 forms, for example, a display image specifically indicating a vortex of a blood flow within a B-mode image related to a cross section within the heart and a display image indicating a distribution of the velocity vectors or a streamline obtained by the distribution of the velocity vectors within a B-mode image. The display image formed by the display image forming unit 60 is displayed on a display unit 62.

A control unit 70 controls the whole ultrasonic diagnostic device illustrated in FIG. 1. The ultrasonic diagnostic device of FIG. 1 may preferably include an operation device, such as a mouse, a keyboard, a trackball, a touch panel, or a joy stick. An instruction received by a user via the operation device is also reflected in the whole control performed by the control unit 70.

Among the elements (each unit designated by a reference numeral) illustrated in FIG. 1, the transmitter and receiver unit 12, the ultrasound image forming unit 20, the Doppler processing unit 30, the velocity vector computation unit 40, the vortex detection unit 50, and the display image forming unit 60 may be implemented by using hardware such as an electrical and electronic circuit or a processor, for example, and a device such as a memory may be used for the implementation. A specific example of the display 62 may include a liquid crystal display, for example. The control unit 70 can be implemented by a cooperation of hardware such as a CPU, a processor, or a memory, and software (program) which regulates the operation of the CPU or the processor.

The ultrasonic diagnostic device illustrated in FIG. 1 is summarized as above. A specific example regarding vortex detection by the ultrasonic diagnostic device of FIG. 1 will now be described in detail. In the following description concerning the elements (sections denoted by reference numerals) illustrated in FIG. 1, the reference numerals in FIG. 1 will be used.

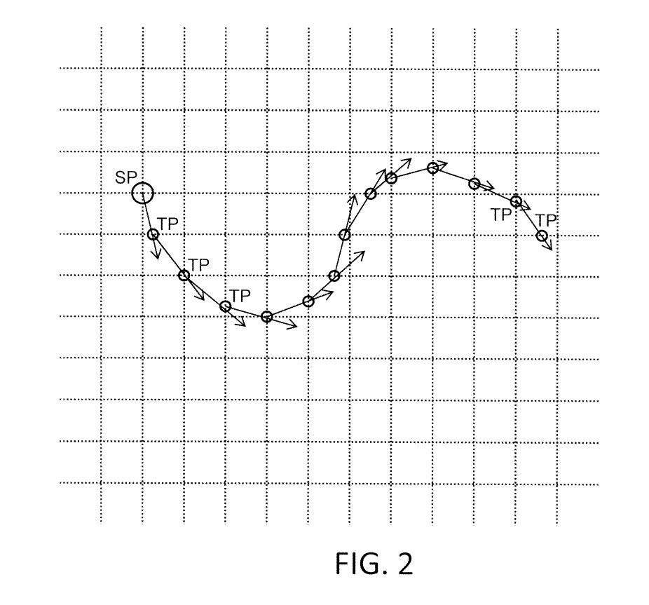

FIG. 2 is a diagram for explaining specific example processing for tracking the flow of a blood flow. The vortex detection unit 50, for each of a plurality of start points, tracks the flow of fluid starting from each start point SP in accordance with a distribution of the two-dimensional velocity vectors. FIG. 2 only shows a single start point SP as a representative example.

The vortex detection unit 50 starts tracking, from a start point SP, in the direction of a velocity vector at the location of the start point SP (an arrow in FIG. 2) to search a tracking point TP. The tracking point TP is searched on an operation grid in a lattice shape, for example, shown by dashed lines. Upon search of a tracking point TP on the operation grid, tracking is continued in the direction of a velocity vector at the tracking point TP, to search the next tracking point TP.

When a velocity vector does not exist at the location of the tracking point TP, an interpolated vector is obtained by interpolation processing, for example, based on a plurality of velocity vectors which have already been calculated near the tracking point TP, and is used as a velocity vector at the tracking point TP.

As illustrated in FIG. 2, the tracking points TP are thus sequentially searched in accordance with the distribution of velocity vectors, starting from the single start point SP, and the flow of a blood flow is tracked. Further, by connecting the start point SP and the plurality of tracking points TP which are adjacent with each other by a straight line or a curved line, a streamline in a polygonal line or a curved line is formed.

The vortex detection unit 50 places a plurality of start points SP within a region of interest which is a diagnosis target, such as a whole region within the heart cavity of the heart, and tracks the flow of a blood flow from each start point SP to form a streamline.

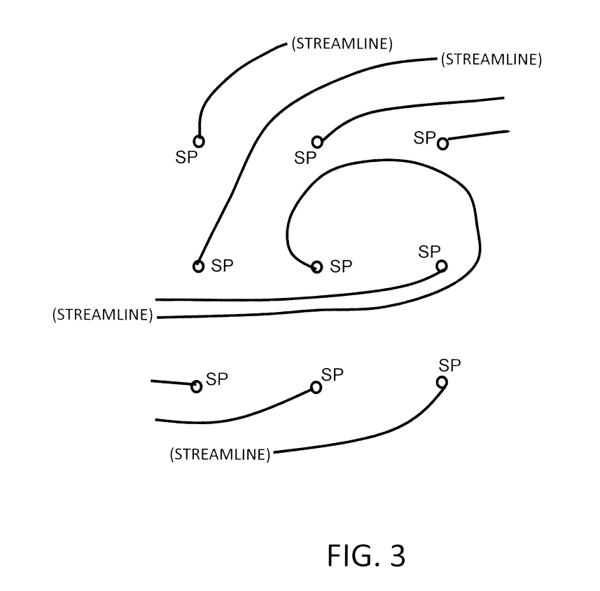

FIG. 3 is a diagram illustrating a specific example arrangement of a plurality of start points SP. The vortex detection unit 50 discretely places a plurality of start points SP in a lattice, as illustrated in FIG. 3, for example, and forms a streamline (solid curved line) for each start point SP. The size of a lattice in which a plurality of start points SP are arranged and the intervals of the lattice (intervals among the plurality of start points SP) may preferably be variable. Upon formation of the streamlines, the vortex detection unit 50 determines, based on the streamline obtained from each start point SP, whether or not the flow of a blood flow starting from the start point SP is a vortex.

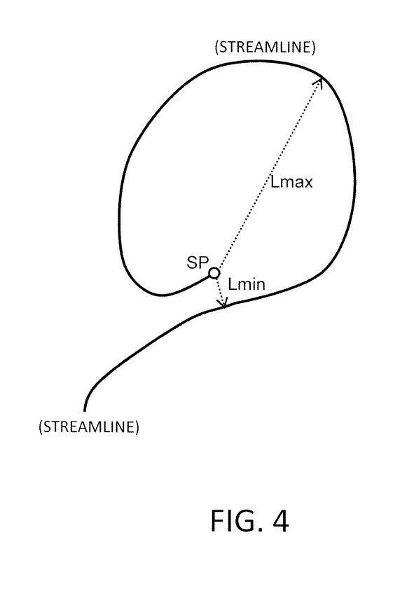

FIG. 4 is a diagram for explaining a specific example regarding determination of a vortex. FIG. 4 illustrates only a single start point SP as a representative example and shows a stream line obtained from the start point SP in a solid line. The vortex detection unit 50 determines, in accordance with a recurrence condition based on a distance from the start point SP to a point on the streamline, whether or not the streamline obtained from the start point SP is a vortex.

Specifically, a distance L from the start point SP to each of a plurality of measuring points on the streamline is calculated, and the maximum distance value Lmax and the minimum distance value Lmin are searched along the streamline. For example, within a search range which is set from the start point SP to a predetermined length of the streamline, the maximum distance value Lmax is first searched, and then the minimum distance value Lmin is searched behind the maximum distance value Lmax (in the direction away from the start point SP on the streamline). The vortex detection unit 50 determines that the streamline obtained from the start point SP is a vortex when the ratio of the minimum distance value Lmin with respect to the maximum distance value Lmax (Lmin/Lmax) is equal to or less than a threshold value (0.4, for example).

The distance L may be calculated using any reference point other than the start point SP of a streamline. For example, a reference point may be set near the start point SP or near the streamline to measure the distance L from the reference point to a measurement point on the streamline. Also, the recurrence condition based on the distance L is merely one specific example regarding determination of a vortex, and determination of a vortex may be performed based on other evaluation values associated with the streamline.

The vortex detection unit 50 determines, for each of a plurality of streamlines (see FIG. 3, for example) obtained from a plurality of start points SP, whether or not the streamline (flow of a blood flow) is a vortex. On determining that a streamline (a flow of fluid) which is tracked from each start point SP is a vortex, the vortex detection unit 50 confirms a flow outside that vortex and determines the outer edge of the vortex.

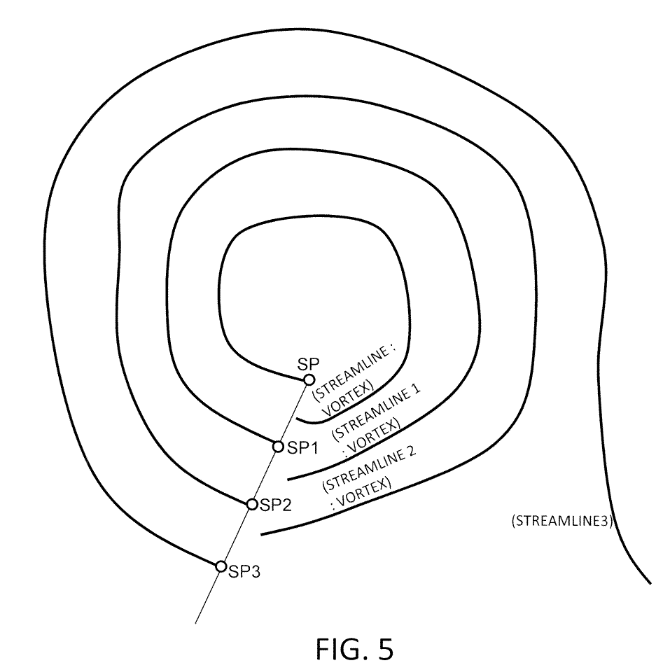

FIG. 5 is a diagram for explaining specific example processing for determining the outer edge of a vortex. In the specific example illustrated in FIG. 5, a streamline obtained from the start point SP is a vortex. Determining that the streamline obtained from the start point SP is a vortex, the vortex detection unit 50 shifts the start point SP toward outside the vortex to confirm a streamline (a flow of fluid) outside the vortex.

As illustrated in FIG. 5, for example, the vortex detection unit 50 shifts the start point SP toward outside the vortex to set a start point SP1, and determines whether or not a streamline 1 obtained from the start point SP1 is a vortex (see FIG. 4). If the streamline 1 is a vortex, the vortex detection unit 50 further shifts the start point SP1 toward outside the vortex to set a further start point SP2, and determines whether or not a streamline 2 obtained from the start point SP2 is a vortex (see FIG. 4). If the streamline 2 is also a vortex, the vortex detection unit 50 further shifts the start point SP2 toward outside the vortex to set a still further start point SP3, and determines whether or not a streamline 3 obtained from the start point SP3 is a vortex (see FIG. 4).

The vortex detection unit 50 thus determines whether or not a streamline is a vortex while shifting the start point SP toward outside the vortex. On confirming that the streamline 3 obtained from the start point SP3 is not a vortex, the vortex detection unit 50 determines the streamline 2 obtained from the start point SP2, which is the outermost streamline that is confirmed to be a vortex, as the outermost vortex. Then, based on the streamline 2, the outer edge of the vortex is determined. Specifically, the start point SP2 and the shortest distance point (a measurement point of the minimum distance value Lmin in FIG. 4) from the streamline 2 are connected with a straight line, and a closed curved line formed by the straight line and the streamline 2 is determined as the outer edge of the vortex. The vortex detection unit 50 may further search a center point of the vortex.

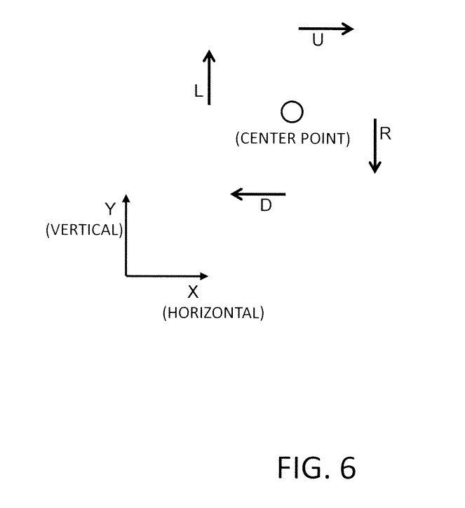

FIG. 6 is a diagram illustrating a specific example center point of a vortex. A plurality of velocity vectors enclose a noted point within a vortex, and, among these velocity vectors, opposing velocity vectors are directed in opposite directions. In this case, the vortex detection unit 50 determines that the noted point is the center point of the vortex.

More specifically, FIG. 6 illustrates that, concerning a noted point within a vortex detected in a two-dimensional plane, a velocity vector U and a velocity vector D which are adjacent to each other in the vertical direction (Y-axis direction) with respect to the noted point are directed in opposite directions and simultaneously a velocity vector L and a velocity vector R which are adjacent to each other in the horizontal direction (X-axis direction) with respect to the noted point are directed in opposite directions. In this case, the vortex detection unit 50 determines that the noted point is the center point of the vortex.

The vortex detection unit 50 determines, for each of a plurality of start points SP (see FIG. 3), whether or not each of a plurality of streamlines obtained from each start point SP is a vortex, and searches for a center point of a vortex when a streamline is determined as a vortex. When a plurality of vortexes which are detected have a center point at the same location, these vortexes are regarded as the same vortexes and treated as one group. Then, a vortex having the largest area, for example, among the plurality of vortexes having the same center point, is selected as a vortex corresponding to the center point.

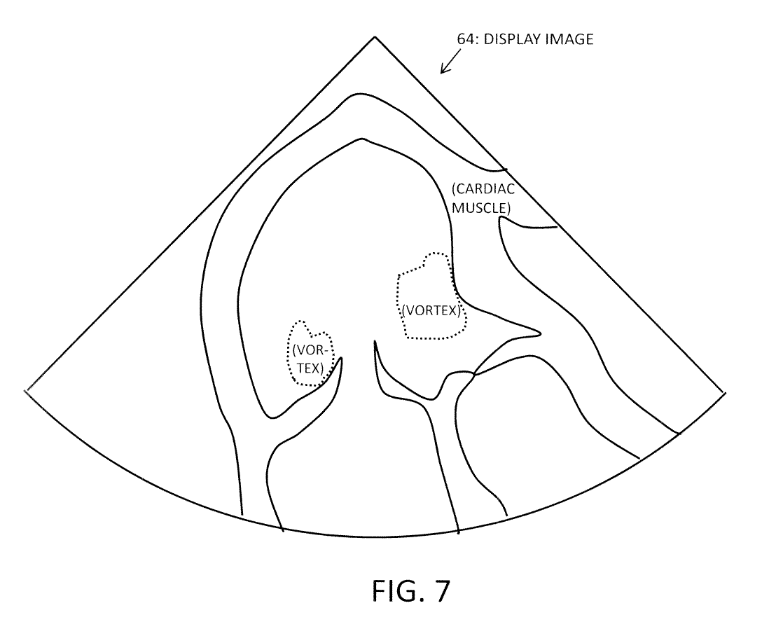

FIG. 7 is a diagram illustrating a specific example display of vortexes. A display image 64 illustrated in FIG. 7 is a specific example image formed by the display image forming unit 60, and specifically indicates a vortex within a blood flow detected by the vortex detection unit 50 in an ultrasound image indicating a cross section within the heart which is formed in the ultrasound image forming unit 20. For example, the outer edge of a vortex obtained by the vortex detection unit 50 is indicated within the display image 64. The display image 64 illustrated in FIG. 7 indicates, in dashed lines, outer edges of two vortexes. A user (examiner) such as a doctor can visually recognize the location and size of the vortexes from the display image 64. Further, diagnosis information concerning the vortex, including coordinates of a center point of the vortex or an area of the vortex (the area of a space enclosed by the outer edge), for example, may also be indicated by numerical values, for example, so that the user such as a doctor can evaluate a vortex quantitatively.

Further, the distribution of velocity vectors at each location which is indicated by an arrow may be shown within the display image 64 or a known color Doppler image may be displayed within the display image 64.

An ultrasonic diagnostic device directed to one or more aspects of the invention is described above. At least one of the velocity vector computation unit 40, the vortex detection unit 50, and the display image forming unit 60 illustrated in FIG. 1 may be implemented by a computer and the computer may be caused to function as a fluid information processor, for example.

The embodiment described above is described only for the illustrative purpose and will not limit the scope of the invention. The invention may encompass various modifications within a range which does not depart from the gist thereof.

### REFERENCE SIGNS LIST

10 probe, 12 transmitter and receiver unit 20 ultrasound image forming unit 30 Doppler processing unit 40 velocity vector computation unit, 50 vortex detection unit 60 image display forming unit, 70 control unit.

1. An ultrasonic diagnostic device, comprising:
a probe configured to transmit and receive an ultrasonic wave;
a transmitter and receiver unit configured to control the probe to obtain a received signal of an ultrasonic wave from within an organism;
a vector computation unit configured to obtain, based on the received signal of an ultrasonic wave, a distribution of a motion vector concerning fluid within the organism; and
a vortex detection unit configured to track a flow of fluid based on the distribution of a motion vector and, based on the flow of the fluid satisfying a recurrence condition, detect a vortex within the fluid.

2. The ultrasonic diagnostic device according to claim 1, wherein
the vortex detection unit is configured to track, for each of a plurality of start points, a flow of fluid in accordance with the distribution of a motion vector from each start point and determine, based on the flow of fluid being tracked from each start point satisfying the recurrence condition, that the flow is a vortex.

3. The ultrasonic diagnostic device according to claim 2, wherein
the vortex detection unit is configured to track the flow of fluid from each start point to obtain a streamline and determine whether or not the flow is a vortex by using the streamline, in accordance with a recurrence condition based on a distance between the start point and a point on the streamline.

4. The ultrasonic diagnostic device according to claim 3, wherein
the vortex detection unit is configured to determine, based on a ratio of a minimum value of the distance from each start point to a point on the streamline with respect to a maximum value of the distance, whether or not the streamline obtained from the start point is a vortex.

5. The ultrasonic diagnostic device according to claim 4, wherein
the vortex detection unit is configured to determine that the streamline obtained from the start point is a vortex, based on the ratio of the minimum value of the distance from each start point to a point on the streamline with respect to the maximum value of the distance being a threshold value or less.

6. The ultrasonic diagnostic device according to claim 2, wherein
the vortex detection unit is configured to further determine, when the flow of fluid being tracked from each start point is a vortex, whether or not a flow of fluid being tracked from each of a plurality of start points outside the vortex is a vortex, and determine an outer edge of the vortex based on a flow of fluid obtained from an outermost start point which is determined to correspond to a vortex.

7. The ultrasonic diagnostic device according to claim 3, wherein
the vortex detection unit is configured to further determine, when the flow of fluid being tracked from each start point is a vortex, whether or not a flow of fluid being tracked from each of a plurality of start points outside the vortex is a vortex, and determine an outer edge of the vortex based on a flow of fluid obtained from an outermost start point which is determined to correspond to a vortex.

8. The ultrasonic diagnostic device according to claim 1, wherein
the vortex detection unit is configured to determine a noted point within a vortex as a center point of the vortex, based on opposing motion vectors, among a plurality of motion vectors enclosing the noted point, being directed to opposite directions.

9. The ultrasonic diagnostic device according to claim 8, wherein
the vortex detection unit is configured to determine a noted point detected within a two-dimensional plane as a center point of a vortex, based on motion vectors adjacent to each other in a vertical direction with respect to the noted point being directed in opposite directions and motion vectors adjacent to each other in a horizontal direction with respect to the noted point being directed in opposite directions.

10. The ultrasonic diagnostic device according to claim 8, wherein
the vortex detection unit is configured to determine, upon detecting a plurality of vortexes having a center point at an identical location, a largest vortex among the plurality of vortexes as a vortex corresponding to the center point.

11. The ultrasonic diagnostic device according to claim 3, wherein
the vortex detection unit is configured to determine a noted point within a vortex as a center point of the vortex, based on opposing motion vectors among a plurality of motion vectors enclosing the noted point being directed in opposite directions.

12. The ultrasonic diagnostic device according to claim 11, wherein
the vortex detection unit is configured to determine a noted point detected within a two-dimensional plane as a center point of a vortex, based on motion vectors adjacent to each other in a vertical direction with respect to the noted point being directed in opposite directions and motion vectors adjacent to each other in a horizontal direction with respect to the noted point being directed in opposite directions.

13. The ultrasonic diagnostic device according to claim 11, wherein
the vortex detector is configured to determine, on detecting a plurality of vortexes having a center point at an identical location, a largest vortex among the plurality of vortexes as a vortex corresponding to the center point.

14. A fluid information processor comprising:
a vector computation unit configured to obtain a distribution of a motion vector concerning fluid within an organism based on a received signal of an ultrasonic wave; and
a vortex detection unit configured to track a flow of the fluid based on the distribution of a motion vector and detect a vortex within the fluid based on the flow of the fluid satisfying a recurrence condition.
